(19) United States
(12) Patent Application Publication
(10) Pub. No.: US 2026/0044017 A1
(43) Pub. Date: Feb. 12, 2026

(54) CONICAL LENS FOR EYEWEAR

(71) Applicant: Oakley, Inc., Foothill Ranch, CA (US)

(72) Inventors: Jeremy SWAN, Norco, CA (US); Ryan SAYLOR, Mission Viejo, CA (US)

(73) Assignee: Oakley, Inc., Foothill Ranch, CA (US)

(21) Appl. No.: 19/292,640

(22) Filed: Aug. 6, 2025

Related U.S. Application Data
(60) Provisional application No. 63/680,367, filed on Aug. 7, 2024.

(51) Int. Cl. G02C 7/02; A61F 9/08

(52) U.S. Cl. CPC G02C 7/02; A61F 9/08

(57) ABSTRACT

Embodiments comprise a lens for an eyewear. The lens comprises a portion of a conical surface configured to be located in the path of a wearer's field of vision. The portion of the conical surface has a first side edge and a second side edge. The portion of the conical surface extends arcuately from the first side edge to the second side edge. The portion of the conical surface has an upper edge and a lower edge. The portion of the conical surface extends from the upper edge to the lower edge. A rate of change of a lens normal from the upper edge to the lower edge is constant. The lens normal is measured along an axis that intersects the conical surface at a pupillary distance.

### CROSS REFERENCE TO RELATED PATENT APPLICATION

This application claims the benefit of U.S. Provisional Patent Application No. 63/680,367, entitled “CONICAL LENS FOR EYEWEAR,” filed on Aug. 7, 2024. The entire content of the above referenced application is incorporated by reference herein in its entirety.

### BACKGROUND

Unitary lens systems provide a full side-to-side range of vision and good lateral eye protection. However, known unitary lens systems provide limited vertical and horizontal field of visions. In addition, lenses may suffer from internal fogging and limited ventilation. Thus, notwithstanding the many advances in lens systems, there is a continuing need for a lens and an eyewear having enhanced field of view that is suitable for all purposes, such as, for example, for indoor use, driving, or select sporting activities.

### BRIEF DESCRIPTION OF THE DRAWINGS

Aspects of this disclosure are best understood from the following detailed description when read with the accompanying figures. It is noted that, in accordance with the common practice in the industry, various features are not drawn to scale. In fact, the dimensions of the various features may be arbitrarily increased or reduced for clarity of discussion, except where noted otherwise.

FIG. 1A is a schematic that illustrates a perspective view of an eyewear configured to provide an enhanced field of view, according to some embodiments.

FIG. 1B is a schematic that illustrates a side view of an eyewear configured to provide an enhanced field of view, according to some embodiments.

FIG. 1C is a schematic that illustrates a perspective view of a lens configured to provide an enhanced field of view, according to some embodiments.

FIG. 2A is a schematic that illustrates a perspective view of a shield, according to some embodiments.

FIG. 2B is a schematic that illustrates a perspective view of a shield, according to some embodiments.

FIGS. 3A-3G are schematics that illustrate field of view (FOV) measurements, according to some embodiments.

FIG. 4 is a schematic that illustrates a target used in FOV measurements, according to some embodiments.

FIG. 5 is a schematic that illustrates FOV measurements for a plurality of eyewear, according to some embodiments.

FIG. 6A is a schematic that illustrates a horizontal slice of a lens, according to some embodiments.

FIG. 6B is a schematic that illustrates wrap angles for a lens, according to some embodiments.

FIG. 7A is a schematic that illustrates a perspective view of a differential offset measurement apparatus, according to some embodiments.

FIG. 7B is a schematic that illustrates a front view of the differential offset measurement apparatus, according to some embodiments.

FIG. 7C is a schematic that illustrates a side view of the differential offset measurement apparatus, according to some embodiments.

Illustrative embodiments will now be described with reference to the accompanying drawings. In the drawings, like reference numerals generally indicate identical, functionally similar, and/or structurally similar elements.

### DETAILED DESCRIPTION

The following disclosure provides many different aspects (also referred to herein as embodiments or examples) for implementing different features of the provided subject matter. Specific examples of components and arrangements are described below to simplify the present disclosure. These are, of course, merely examples and are not intended to be limiting. For example, the formation of a first feature over a second feature in the description that follows may include embodiments in which the first and second features are formed in direct contact, and may also include embodiments in which additional features may be formed between the first and second features, such that the first and second features may not be in direct contact. As used herein, the formation of a first feature on a second feature means the first feature is formed in direct contact with the second feature. In addition, the present disclosure may repeat reference numerals and/or letters in the various examples. This repetition does not in itself dictate a relationship between the various embodiments and/or configurations discussed.

The term “lens” as used herein is used to broadly refer to an optical component. For example, eyeglass/sunglass lenses, vision shields, visors, and the like are included in the term “lens” or “lens for eyewear.” The term “non-corrective” as used herein indicates a lack of optical power as understood for prescription lenses.

Spatially relative terms, such as “beneath,” “underlying,” “underneath,” “below,” “lower,” “above,” “over,” “upper,” “lower,” and the like may be used herein for ease of description to describe one element or feature's relationship to another element(s) or feature(s) as illustrated in the figures. The spatially relative terms are intended to encompass different orientations of the device in use or operation in addition to the orientation depicted in the figures. The device may be otherwise oriented (rotated 90 degrees or at other orientations) and the spatially relative descriptors used herein may likewise be interpreted accordingly.

It is noted that references in the specification to “one embodiment,” “an embodiment,” “an example embodiment,” “exemplary,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

It is to be understood that the phraseology or terminology herein is for the purpose of description and not of limitation, such that the terminology or phraseology of the present specification is to be interpreted by those skilled in relevant art(s) in light of the teachings herein.

In some embodiments, the terms “about” and “substantially” can indicate a value of a given quantity that varies within 5% of the value (e.g., ±1%, ±2%, ±3%, ±4%, or ±5% of the value).

The terms “typical wearer,” “typical user,” and the like as used herein may refer to a median user in general, a median user according to a demographic, or a user having physical dimensions conforming to a standard or a well-known database of human measurements. For example, a typical eyewear wearer may be one having physical dimensions that conform to European Standards (EN), American National Standards Institute (ANSI), or anthropometric surveys, among others.

Additionally, although particular embodiments may be disclosed or shown in the context of particular types of eyewear, such as unitary lens eyeglasses, dual lens eyeglasses, eyeglasses having partial, full, or no orbitals, goggles, sunglasses, eyewear with earstems, eyewear with partial earstems, eyewear without earstems, and the like, it is to be appreciated that embodiments of the present invention may be used in any type of headworn support. For example, lens embodiments may be integrated into or attached to an item of headgear, such as a bicycle, skateboarding, snow, flight, sport, or other type of helmet with a vision shield, a visor, a hat, a headband, face mask, balaclava, breaching shield, or any other any headworn article that may support one or more lenses in the wearer's field of view. In some embodiments, the lens may be detachable from the headworn article so that the lens may be removed or replaced without damaging the headworn article.

As used herein, the term “disposed,” as used for example in “a first layer is disposed over a second layer,” means that the first layer is either directly placed against the second layer's surface, or that the first layer is indirectly placed over the second layer's surface with at least a third layer in between.

As used herein, the term “coupled,” as used for example in “a first layer is coupled to a second layer” means that the first layer is disposed over the second layer (as “disposed” is defined above), or that the first layer is integrated into the second layer.

It is to be understood that the frame of reference (e.g., the axes and planes) described herein are discussed in connection within standard contexts with a user's head in an upright vertical position, For example, an anatomical superior-inferior axis is generally referred to in connection with a vertical axis, the anatomical medio-lateral axis is generally referred to in connection with a horizontal plane. This can be measured, for example, on a standard headform such as, but not limited to, an Alderson headform, an EN168 headform, a CSA Z262.2-14 headform, or any other standard headform. However, it is also understood that the frame of reference described herein may be shifted in other contexts.

Despite the many advances of eyewear lenses, there is a continuing need for a lens having excellent optical qualities and providing enhanced field of view while at the same time providing a configuration that allows for adequate ventilation, maximum comfort and safety to the wearer, reduced fogging, and/or attachment to specific headgear. Described herein is a lens having a shape configured to improve venting, minimize fogging, and improve the field of view of the wearer. In some aspects, the lens is configured to conform to the face of the wearer (e.g., close to the face of a wearer).

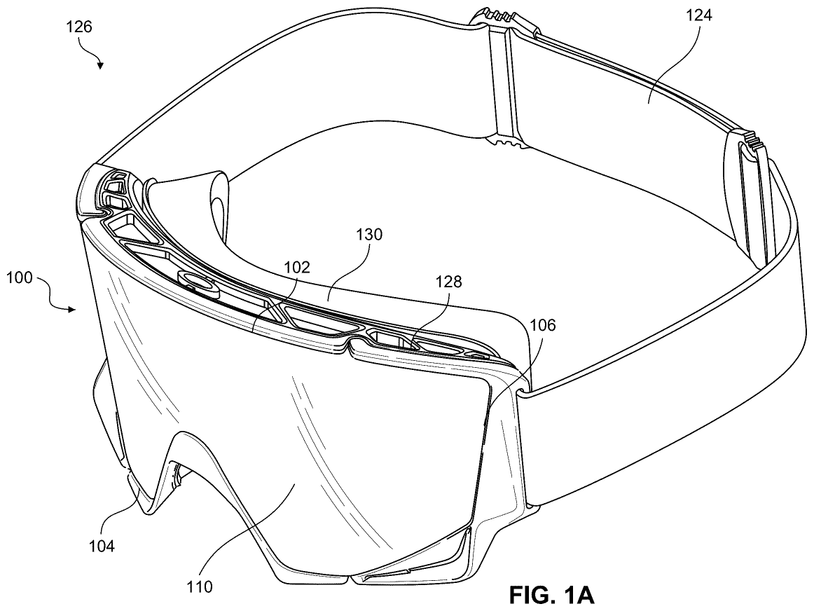

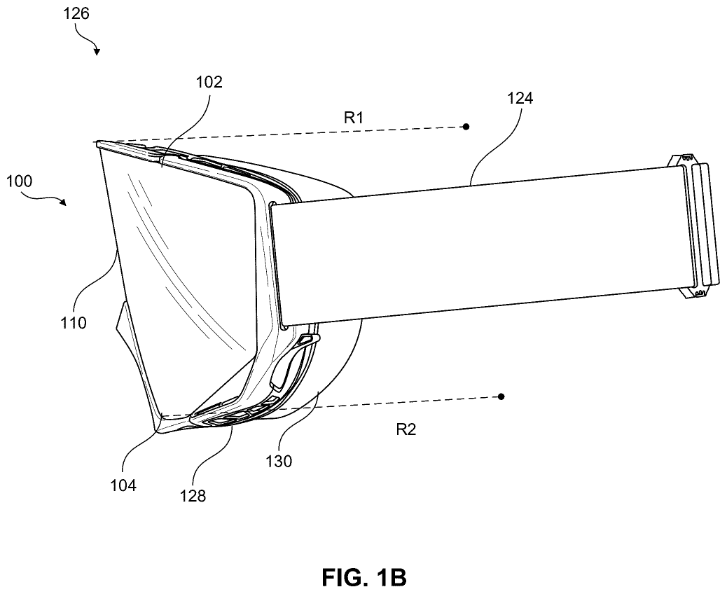

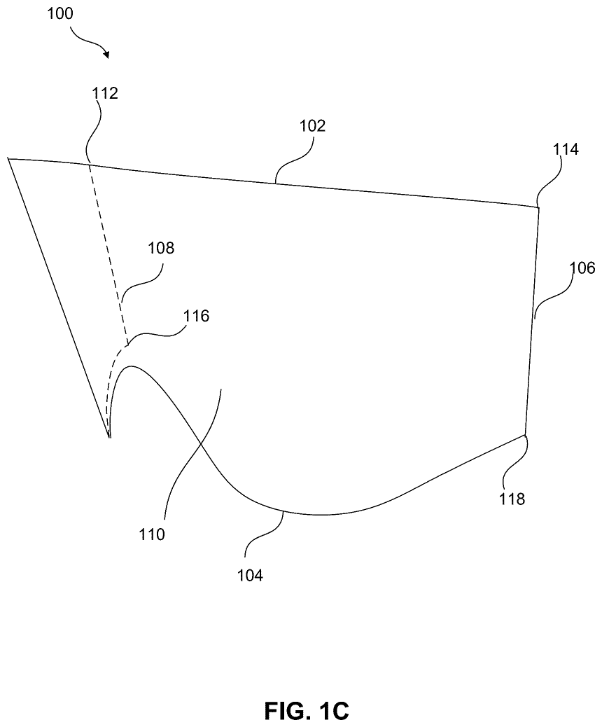

FIG. 1A illustrates a perspective view of an eyewear 126 configured to provide field of view enhancement, according to some embodiments. FIG. 1B illustrates a side view of eyewear 126. Eyewear 126 can include a lens 100 and a mounting frame 128 configured to support lens 100. In some embodiments, the mounting frame or support can be configured to be supported on a head or user for positioning lens 100 in the path of a wearer's field of vision. Eyewear 126 can be a goggle, as shown for example in FIG. 1A having a strap 124 for supporting mounting frame 128 on a head of a wearer. In another aspect, eyewear 126 can be eyeglasses and include left and right earstems (not shown) to support the eyewear on the wearer's head. In FIG. 1A, eyewear 126 is shown to include a unitary lens 100 wherein a single lens is in front of both eyes of wearer. However, it is to be understood that eyewear 126 can include more than a single lens (e.g., a dual-lens eyewear with one lens in front of each eye of user). Further, eyewear 126 can include a second inner lens which may be separated from first lens 100 by a space. The space may be filled with air or another gas to provide an air gap for thermal insulation.

In some embodiments, mounting frame 128 can include a contact member 130 which contacts the user's face. In some embodiments, contact member 130 can be formed from a permeable material such as foam or a fluid impermeable material such as rubber or silicone. In some aspects, eyewear 126 can be frameless (no orbital), and contact member 130 may be supported directly on a rear side of lens 100. In such aspects, lens 100 can be designed with sufficient structural rigidity to serve as the main structural support of eyewear assembly 100. Further, lens 100 may include attachment points for strap 124 and/or earstems. In some aspects, lens 100 can be modular and interchangeable. This can beneficially allow a user to swap between lenses and/or frames as desired. In other aspects, lens 100 may not be interchangeable. In some aspects, mounting frame 128 may comprise a goggle frame, e.g., a snow/ski goggle frame, a motorcycle goggle frame, etc. Examples of frameless eyewear are described in U.S. Pat. Nos. 11,679,033 and 11,526,025, which are both incorporated herein by reference in their entireties.

In some aspects, mounting frame 128 can be configured to surround at least a portion (e.g., for semi-rimless eyewear 126) or an entirety of the periphery of lens 100 (full-rimmed eyewear 126). Mounting frame 128 is shown extending along the entirety of periphery of lens 100 in FIG. 1A; however, it is to be understood that mounting frame 128 can extend along a bottom periphery of lens 100, can extend along a top periphery of lens 100 in combination with or in lieu of the portion which extends along a bottom periphery of lens 100, or can extend along one or two side peripheries of lens 100. In some aspects, mounting frame 128 can include ear supports (earstems) that are directly attached to lens 100 (e.g., for rimless or semi-rimless eyewear 126 embodiments).

Eyewear 126 can be of any type, including general-purpose eyewear, special-purpose eyewear, sunglasses, driving glasses, sporting glasses, goggles (including for sport or safety), indoor eyewear, outdoor eyewear, eyewear incorporated into headgear (such as visors for helmets), vision-correcting eyewear, prescription and non-prescription eyeglasses, color vision deficiency eyewear, contrast-enhancing eyewear, chroma-enhancing eyewear, color-enhancing eyewear, color-altering eyewear, gaming eyewear, eyewear designed for another purpose, or eyewear designed for a combination of purposes. In some embodiments, lenses and frames of many other shapes and configurations may be used for eyewear 126. For example, eyewear 126 can have a single lens, such as in a goggle or visor. In some aspects, eyewear 126 may include a dual lens. It should be noted that eyewear 126 shown in FIGS. 1A-1C is not drawn to scale but is drawn to more easily illustrate certain aspects of eyewear 126.

FIG. 1C illustrates a perspective view of lens 100 of eyewear 126 configured to provide field of view enhancement, according to some embodiments. Lens 100 can have a lens body 110. The anterior surface and/or the posterior surface of lens body 110 can conform to a surface of a cone such that a radius of curvature along a horizontal direction is uniform in each horizontal plane (e.g., along the anatomical medio-lateral axis with respect to a EN headform), while the radius of curvature along the horizontal direction is increased or decreased from one horizontal plane to the next according to a constant slope.

Lens 100 can include an upper edge 102, a lower edge 104, a first side edge 106, a second side edge 108, and lens body 110. Upper edge 102 has a first end 112 and a second end 114. Lower edge 104 has a first end 116 and a second end 118. In some aspects, lens body 110 extends linearly from upper edge 102 to lower edge 104. In some aspects, lens body 110 extends arcuately from first side edge 106 to second side edge 108. In some aspects, lower edge 104 is angled towards a wearer's cheekbones as compared to upper edge 102. In some aspects, lens 100 may be formed from a frustoconical surface. For example, a portion of upper edge 102 conforms to a first imaginary arc having a radius R1 and a portion of lower edge 104 conforms to a second imaginary circular arc having a radius R2 (shown in FIG. 1B), where radius R2 is different from radius R1. In some aspects, radius R2 is less than radius R1. In some aspects, a horizontal distance from first end 112 of upper edge 102 to second end 114 of upper edge 102 is greater than a horizontal distance from first end 116 of lower edge 104 to second end 118 of lower edge 104. In some aspects, a wider upper edge 102 provides the advantage of improved venting while the narrower lower edge 104 (close to the cheeks) provides the advantage of increased field of view.

In some aspects, radius R1 may be from about 70 mm to about 130 mm, from about 75 mm to about 125 mm, from about 80 mm to about 120 mm, from about 85 mm to about 115 mm, from about 90 mm to about 110 mm, or from about 95 mm to about 105 mm. In some aspects, R1 may be about 95 mm, 100 mm, or about 105 mm. In some aspects, radius R2 may be from about 70 mm to about 100 mm, from about 75 mm to about 95 mm, from about 80 mm to about 90 mm, or about 83 mm to about 87 mm. In some aspects, R2 may be about 84 mm, about 85 mm, or about 86 mm.

In some aspects, radius R1 may be less than radius R2. For example, radius R1 may be from about 70 mm to about 100 mm, from about 75 mm to about 95 mm, from about 80 mm to about 90 mm, or about 83 mm to about 87 mm. In some aspects, R1 may be about 84 mm, about 85 mm, or about 86 mm. Radius R2 may be from about 70 mm to about 130 mm, from about 75 mm to about 125 mm, from about 80 mm to about 120 mm, from about 85 mm to about 115 mm, from about 90 mm to about 110 mm, or from about 95 mm to about 105 mm.

Lens body 110 can be provided with anterior and posterior surfaces and a thickness therebetween. In some aspects, the thickness can be variable along the horizontal direction, vertical direction, or combination of directions. In some aspects, lens body 110 can have a varying thickness along the horizontal or vertical axis, or along some other direction. In some aspects, the thickness of the lens tapers smoothly, though not necessarily linearly, for a maximum thickness proximate a medial edge to a relatively lesser thickness at a lateral edge. Lens body 110 can have a tapering thickness along the horizontal axis and can be decentered for optical correction. In some aspects, lens body 110 can have a thickness configured to provide an optical correction. In some aspects, lens body 110 can have a substantially uniform thickness.

In some aspects, the thickness across lens body 110 (e.g., at any point on lens body) from the anterior to the posterior surface and normal to at least one of the anterior or posterior surface may range from about 1 mm to about 3 mm, from about 1.2 mm to about 2.8 mm, from about 1.4 mm to about 2.4 mm, from about 1.4 mm to about 2.2 mm. In some aspects, the thickness of lens body 110 measured at a pupillary distance (PD) from a center axis of lens body 110 may be from about 2 mm to about 2.4 mm, from about 2.1 mm to about 2.3 mm, or about 2.2 mm. In some aspects, the thickness of lens body 110 measured at a pupillary distance (PD) from a center of a head may be from about 2 mm to about 2.4 mm, from about 2.1 mm to about 2.3 mm, or about 2.2 mm, measured against the EN headform.

In some aspects, a perpendicular distance between upper edge 102 and lower edge 104 (i.e., measured in a perpendicular direction from at least one of upper edge 102 or lower edge 104) may be from about 80 mm to about 150 mm, from about 85 mm to about 145 mm, from about 90 mm to about 140 mm, from about 100 mm to about 130 mm, from about 105 mm to about 125 mm, from about 115 mm to about 135 mm, from about 120 mm to about 130 mm, or from about 110 mm to about 120 mm. In some aspects, the perpendicular distance may represents the maximal distance measured in a perpendicular direction from at least one of upper edge 102 or lower edge 104.

In some aspects, lens 100 may be formed by or on surface similar to conical surface 110. For example, an opening that corresponds to the wearer's nose may be formed in a lower portion of conical surface 110. In addition, a shape of lower edge may correspond to the contour of a face of the wearer.

A lens for use in eyewear is typically required to comply with safety standards set by market demands or by a regulatory body, for example, a sport organization. While the below description is made primarily in the context of non-corrective eyewear, a person skilled in the art will recognize that similar techniques may be used to improve corrective eyewear as well. Typically, material and thickness are two interrelated safety parameters of lenses for eyewear; for example, a material with high shatter resistance may allow for a thinner lens geometry than another material with a lower shatter resistance.

It is to be appreciated that lens 100 may be designed to be made of lens material commonly used in the art and that the lens material is chosen, based on intended application, for its optical and mechanical properties, for example, low/high refractive indices (e.g., 1.4-1.8), dispersion properties, UV attenuation, and impact resistance properties, among others. The materials may include, for example and without limitation, polycarbonate, CR-39™, TRIVEX™, TRIBRID™, glass, and polymethyl methacrylate (PMMA), among others.

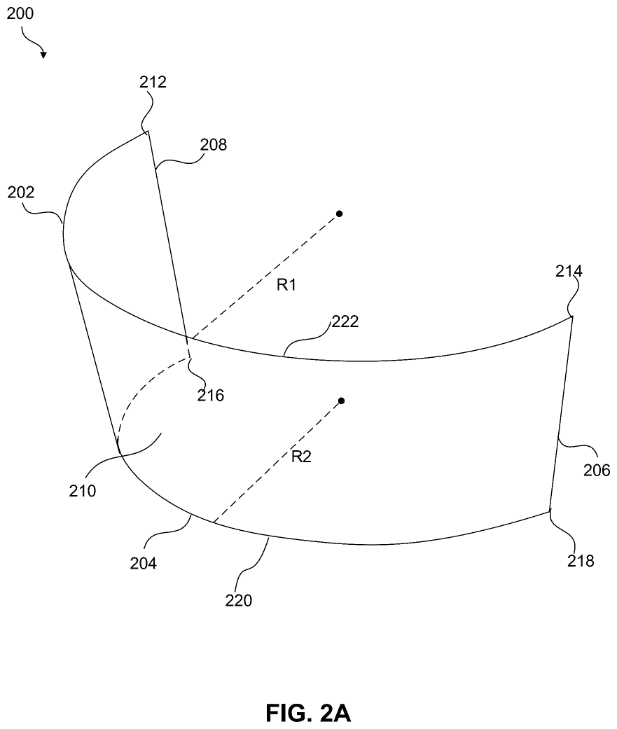

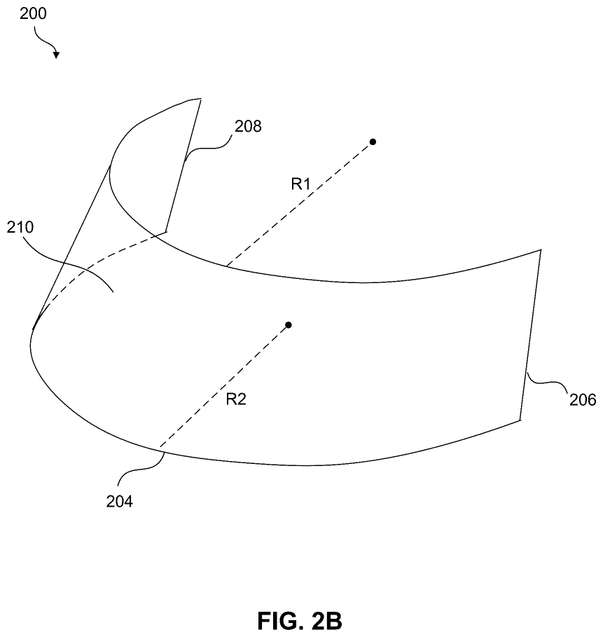

FIG. 2A is a schematic that illustrates a perspective view of a shield 200, according to one embodiment. In some aspects, shield 200 may be coupled to a helmet (e.g., a sport helmet) (not shown). In some aspects, shield 200 may be a face shield or an eye shield. In some aspects, shield 200 may include an upper arcuate edge 202, a lower arcuate edge 204, a first side edge 206, a second side edge 208, and a conical surface 210 (e.g., a lens body having a surface that conforms to a cone). In some aspects, upper arcuate edge 202 and lower arcuate edge 204 may each be oriented in a separate substantially horizontal two-dimensional plane. Upper arcuate edge 202 has a first end 212 and a second end 214. Lower arcuate edge 204 has a first end 216 and a second end 218. In some aspects, upper arcuate edge 202 comprises an arc segment 222 and lower arcuate edge 204 comprises an arc segment 220. In some aspects, the entirety of upper arcuate edge 202 includes arc segment 222. In some aspects, the entirety of lower arcuate edge 204 includes arc segment 220. In some aspects, arc segment 222 and arc segment 220 may be a portion of upper arcuate edge 202 and lower arcuate edge 204, respectively.

Conical surface 210 extends from upper arcuate edge 202 to lower arcuate edge 204 and extends in an arcuate orientation from first side edge 206 to second side edge 208. In some aspects, first side edge 206 and second side edge 208 share common ends with upper arcuate edge 202 and lower arcuate edge 204. Side edge 206 extends from second end 214 of upper arcuate edge 202 to second end 218 of lower arcuate edge 204. Side edge 208 extends from first end 212 of upper arcuate edge 202 to first end 216 of lower arcuate edge 204.

In some aspects, a length of arc segment 220 of lower arcuate edge 204 is less than the length of arc segment 222 of upper arcuate edge 202. For example, arc segments 220, 222 may be defined by an elliptic arc. In some aspects, a semi-minor axis of the ellipse with a portion creating lower arc segment 220 is less than a semi-minor axis of the ellipse with a portion creating upper arc segment 222. In some aspects, arc segments 220, 222 may be circular arcs. In some aspects, a radius R2 of a circle with a portion creating arc segment 220 may be less than a radius R1 of the circle with a portion creating arc segment 222.

In some aspects, radius R1 may be from about 80 mm to about 120 mm, from about 90 mm to about 110 mm, or from about 95 mm to about 105 mm. In some aspects, R1 may be about 95 mm, 100 mm, or about 105 mm. Radius R2 may be from about 75 mm to about 95 mm, from about 80 mm to about 90 mm, or about 83 mm to about 87 mm. In some aspects, R2 may be about 84 mm, about 85 mm, or about 86 mm.

In some aspects, a lens may be formed by or on surface similar to conical surface 210. For example, an opening that corresponds to the wearer's nose may be formed in a lower portion of conical surface 210. In addition, a shape of lower edge may correspond to the contour of a face of the wearer.

FIG. 2B is a schematic that illustrates a perspective view of shield 200, according to another embodiment. In some aspects, a length of arc segment 220 of lower arcuate edge 204 is greater than the length of arc segment 222 of upper arcuate edge 202. The semi-minor axis of the ellipse with a portion creating lower arc segment 220 is greater than the semi-minor axis of the ellipse with a portion creating upper arc segment 222. In some aspects, radius R2 of a circle with a portion creating arc segment 220 may be greater than radius R1 of the circle with a portion creating arc segment 222. Radius R1 may be from about 75 mm to about 95 mm, from about 80 mm to about 90 mm, or about 83 mm to about 87 mm. In some aspects, R1 may be about 84 mm, about 85 mm, or about 86 mm. In some aspects, radius R2 may be from about 80 mm to about 120 mm, from about 90 mm to about 110 mm, or from about 95 mm to about 105 mm. In some aspects, R2 may be about 95 mm, 100 mm, or about 105 mm.

In some aspects, a perpendicular distance between upper arcuate edge 202 and lower arcuate edge 204 may be from about 90 mm to about 140 mm, from about 100 mm to about 130 mm, from about 105 mm to about 125 mm, or from about 110 mm to about 120 mm.

### Example Optical Performance Measurements

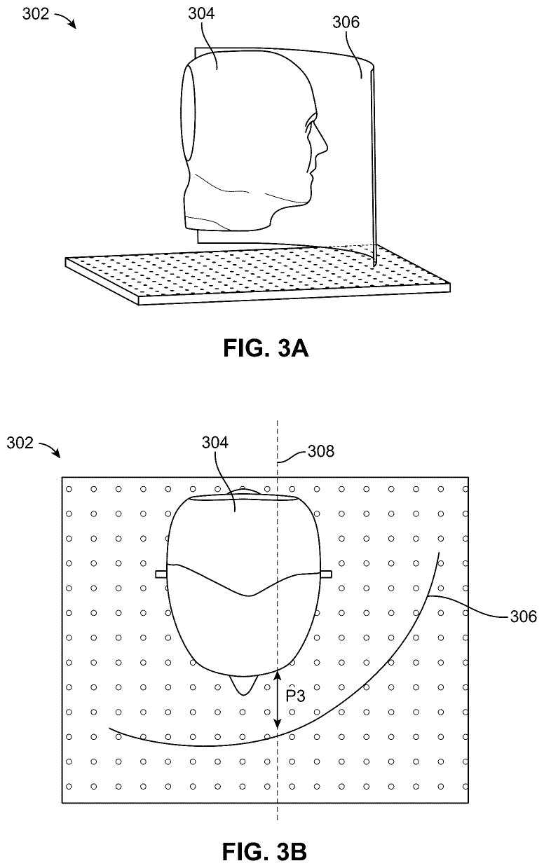

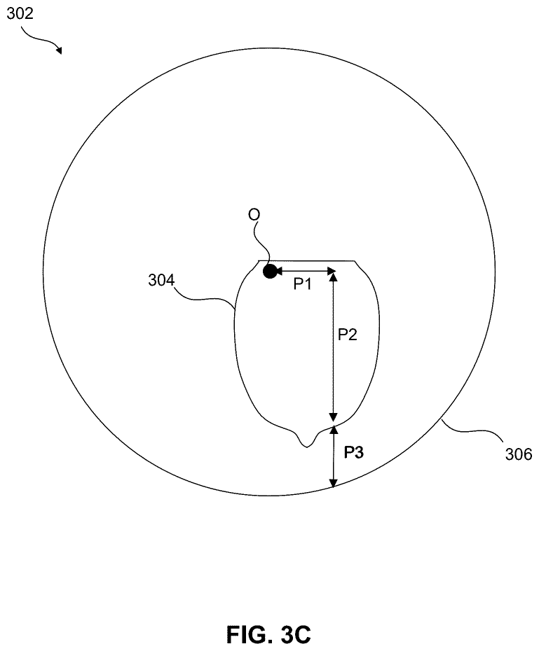

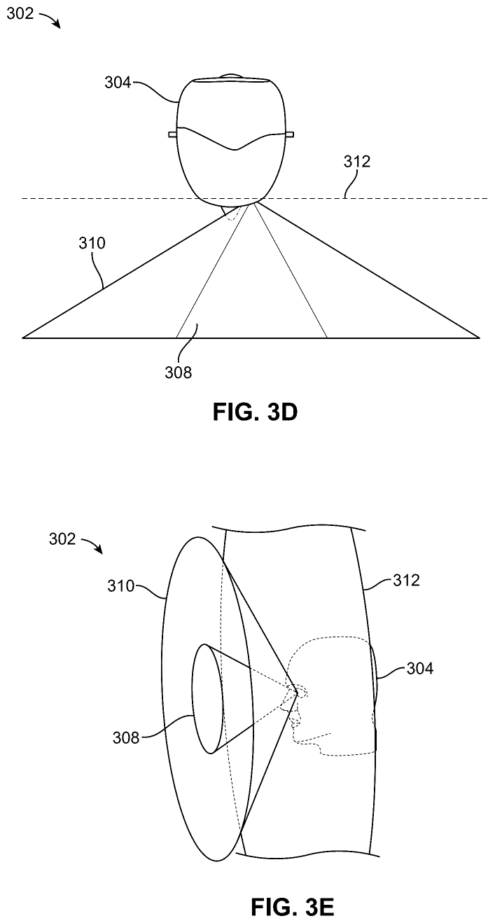

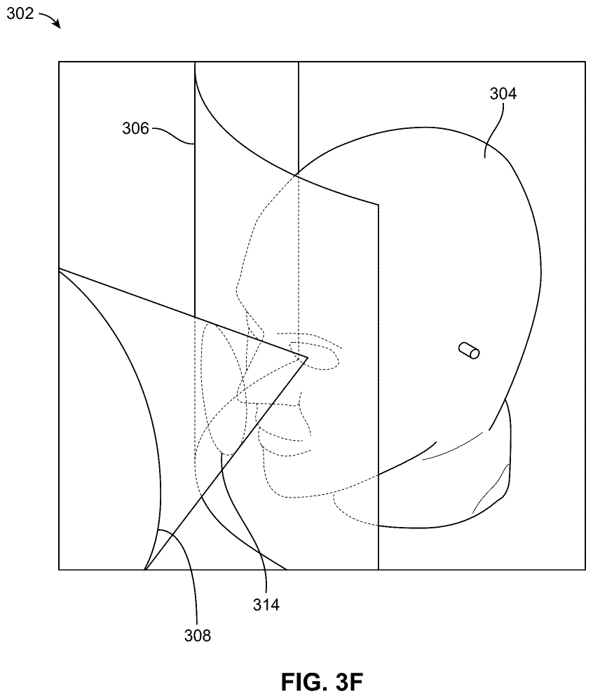

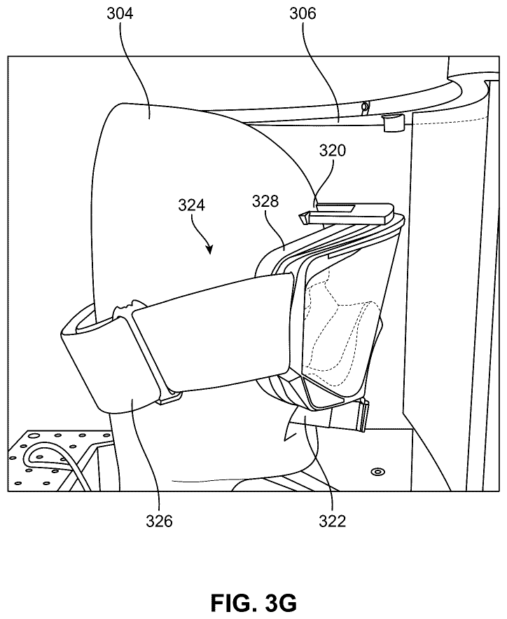

FIGS. 3A-3G illustrate an example field of view (FOV) measurement environment 302, according to an example embodiment. FOV measurement environment 302 may be used to determine a field of view of lens 100, eyewear 126, and/or shield 200. FIG. 3A is a schematic that illustrates a side view of environment 302. FIG. 3B is a schematic that illustrates a top view of FOV measurement environment 302, according to an example embodiment. FOV measurement environment 302 comprises a mounting stage that may correspond to an EN headform 304 and a cylindrical target 306. In some aspects, a distance P3 along axis 308 between an eye center of EN headform 304 and cylindrical target 306 is 73 mm. In some aspects, a surface of cylindrical target 306 conforms to a portion of a surface of an imaginary cylinder (e.g., a cylinder defining a surface of the cylindrical target). In some aspects, a radius of the imaginary cylinder is 243 mm. In some aspects, cylindrical target 306 may be positioned at other distances during the measurements. At such other distances, the actual values of measurement may be different from those provided herein but maintain the same general relationship therebetween.

FIG. 3C is a schematic that illustrates a positioning of EN headform 304 with respect to cylindrical target 306 during the FOV measurements, according to an example embodiment. In some aspects, a light cone may be emanating from the eye center of the EN headform 304. A position of the light source may correspond to the center of the eye. The eye center may be located a horizontal distance P1 and a vertical distance P2 from a center axis O of cylindrical target 306 (e.g., a center of an imaginary cylinder that cylindrical target 306 conforms to a portion of). In some aspects, horizontal distance P1 may be about 72 mm. In some aspects, vertical distance P2 may be about 159 mm.

FIG. 3D is a schematic that illustrates a top view of measurement environment 302 with light cones emanating from the center of the eye of EN headform 304. FIG. 3E is a schematic that illustrates a side view of measurement environment 302 with the light cones emanating from the center of the eye of EN headform 304. The light cones may be used to create a target for measuring the FOV of a lens. In some aspects, a first light cone 308, a second light cone 310, and a third light cone 312 may be used. First light cone 308 may represent a 30° revolved cone from the eye center of EN headform 304. Second light cone 310 may represent a 60° revolved cone from the eye center of EN headform 304 and third light cone 312 may represent a 95° revolved cone from the eye center of EN headform 304.

FIG. 3F is a schematic that illustrates a perspective view of FOV measurement environment 302 to obtain the target for FOV measurements. Each light cone of first light cone 308, second light cone 310, and third light cone 312 is projected onto cylindrical target 306. FIG. 3F shows a first projection 314 of first light cone 308 into cylindrical target 306.

FIG. 3G is a schematic that illustrates a positioning of an eyewear 324 on EN headform 304 during FOV measurements, according to an embodiment. During FOV measurements, a paper or other recording medium may be mounted on cylindrical target 306 to record the projections of the vision cones and a shadow of eyewear 324. Eyewear 324 (e.g., a goggle) may include a strap 326 and a contact member 328. However, during FOV measurements, eyewear 324 is positioned on EN headform 304 without using strap 326 (i.e., no strap tension is applied). During FOV measurements, eyewear 324 may be positioned on EN headform 304 using a first arm 320 and a second arm 322 of EN headform 304. Eyewear 324 may be positioned such that contact member 328 (e.g., foam) just touches a surface of EN headform 304 (i.e., contact but no compression of the foam). Thus, a distance between the center of the eye and the lens of the eyewear under measurement is not affected by the compression of the foam, and the measurements are reliable and consistent when measuring the FOV of different eyewear. This provides the advantage of accurate FOV measurements when measuring the FOV of different eyewear. First arm 320 maintains a vertical positioning of eyewear 324. Horizontal alignment is maintained by aligning a center of eyewear 324 with a center of a nose of EN headform 304.

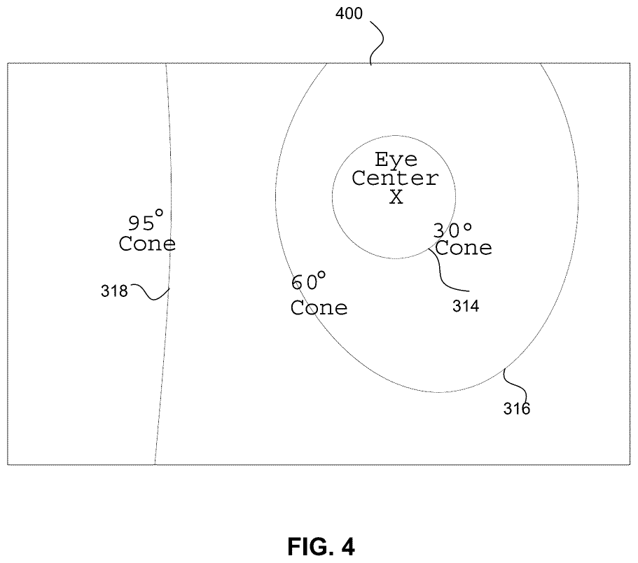

FIG. 4 is a schematic that illustrates a target 400 used in the FOV measurements, according to an example embodiment. Target 400 is obtained using measurement apparatus 302 by projecting each of the vision cones (first light cone 308, second light cone 310, and third light cone 312) onto cylindrical target 306. Target 400 includes first projection 314, a second projection 316, and a third projection 318. As discussed above, first projection 314 is obtained by projecting first light cone 308. Second projection 316 is obtained by projecting second light cone 310 and third projection 318 is obtained by projecting third light cone 312. It is understood that additional projections for other vision cones may be used to quantify the FOV of a lens or an eyewear (e.g., lens 100)

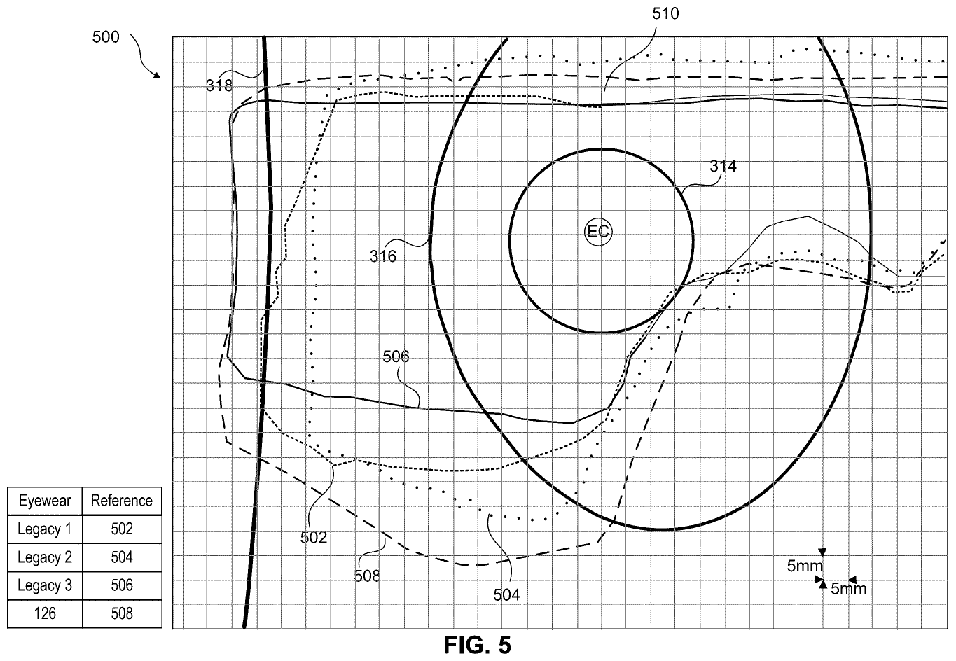

FIG. 5 is a schematic 500 that shows FOV measurements for a plurality of eyewear, according to an example embodiment. In some aspects, a shadow of an eyewear or a lens may be obtained by positioning the eyewear on EN headform 304, as described in relation with FIG. 3G. The shadow of the eyewear is traced on target 400, and represents the extent of the lens on the target. In some aspects, the shadow of the eyewear is obtained using a light source positioned at the center of the eye of EN headform 304. The light source may be, for example, a light emitting diode (LED) positioned at the center of the eye of EN headform 304. FIG. 5 shows a first shadow 502 corresponding to a legacy eyewear 1, a second shadow 504 corresponding to a legacy eyewear 2, a third shadow 506 corresponding to a legacy eyewear 3, and a fourth shadow 508 corresponding to eyewear 126 described herein. In some aspects, the field of view of the various eyewear may be identified by determining an area outside a field of view projection (e.g., first projection 314, second projection 316, and third projection 318). As shown by FIG. 5, fourth shadow 508 corresponding to eyewear 126 shows peripheral gain in the FOV compared to other legacy eyewear.

In some aspects, an area to the left of eye center axis 510 and outside of the projections may be used to quantify the FOV of the eyewear or the lens. In order to determine the area, target 400 may include a grid. In some aspects, a unit dimension of the grid may be, for example and without limitation, 5 mm by 5 mm. Grids having different unit dimensions may also be used. However, when comparing different eyewear, grids having the same unit dimension are typically used.

In some aspects, the area of the shadow corresponding to each eyewear that is outside first projection 314, second projection 316, and third projection 318 and to the left of eye center axis 510 is determined.

In some aspects, an area of a shadow of eyewear 126 or lens 100 outside of a first field view and to the left of eye center axis 510 (first projection 314, shown in FIG. 5 by bolded line 314) is greater than about 300 cm², greater than about 310 cm², greater than about 320 cm², greater than about 330 cm², greater than about 340 cm², greater than about 350 cm², greater than about 360 cm², greater than about 370 cm², or greater than about 380 cm². In some aspects, the area of the shadow of eyewear 126 or lens 100 outside of a first field view (first projection 314) is between about 300 cm² and about 400 cm², between about 310 cm² and about 390 cm², between about 320 cm² and about 380 cm², between about 330 cm² and about 370 cm², or between about 340 cm² and about 360 cm². In some aspects, the area of the shadow outside of a first field view (first projection 314) is about 310 cm², about 320 cm², about 330 cm², about 340 cm², about 350 cm², about 360 cm², about 370 cm², about 380 cm², about 390 cm², or about 400 cm².

In some aspects, an area of a shadow of eyewear 126 or lens 100 outside of a second field of view and to the left of eye center axis 510 (second projection 316, shown in FIG. 5 by bolded line 316) is greater than about 200 cm², greater than about 210 cm², greater than about 220 cm², greater than about 230 cm², greater than about 240 cm², greater than about 250 cm², greater than about 260 cm², greater than about 270 cm², greater than about 280 cm², or greater than about 290 cm². In some aspects, the area is between about 200 cm² and about 300 cm², between about 210 cm² and about 290 cm², between about 220 cm² and about 280 cm², between about 230 cm² and about 270 cm², about 240 cm² and about 260 cm². In some aspects, the area is about 200 cm², about 210 cm², about 220 cm², about 230 cm², about 240 cm², about 250 cm², about 260 cm², about 270 cm², about 280 cm², about 290 cm², or about 300 cm².

In some aspects, an area of a shadow of lens eyewear 126 or lens 100 outside a third field of view and to the left of eye center axis 510 (third projection 318, shown in FIG. 5 by bolded line 318) is greater than about 25 cm², greater than about 30 cm², greater than about 35 cm², greater than about 40 cm², greater than about 45 cm², greater than about 50 cm², greater than about 55 cm², greater than about 60 cm², greater than about 65 cm², greater than about 70 cm², greater than about 75 cm cm², greater than about 80 cm², greater than about 85 cm², greater than about 90 cm², greater than about 95 cm², or greater than about 100 cm².

In some aspects, the area of the shadow of eyewear 126 or lens 100 outside the third field of view is between about 25 cm² and about 100 cm², between about 30 cm² and about 95 cm², between about 35 cm² and about 90 cm², between about 40 cm² and about 85 cm², between about 45 cm² and about 80 cm². In some aspects, the area of the shadow of eyewear 126 or lens 100 outside the third field of view is between about 25 cm² and about 35 cm², between about 27 cm² and about 33 cm², or between about 29 cm² and about 31 cm². In some aspects, the area of the shadow of eyewear 126 or lens 100 is about 28 cm², about 29 cm², about 30 cm², about 31 cm², about 32 cm², or about 33 cm².

Eyewear 126 or lens 100 provides an enhanced FOV compared to conventional eyewear. In some aspects, eyewear 126 or lens 100 provides enhancement in the vertical field of view. In some aspects, eyewear 126 or lens 100 provide enhancement to a lower visual field. As shown in FIG. 5, shadow 508 has the greatest area compared to the shadow of other conventional eyewear in the lower visual field (towards the bottom of schematic 500). Table 1 shows measured areas of various legacy eyewear as compared to lenses and eyewear having an increased FOV as described with respect to embodiments of the present disclosure.

TABLE 1
Measured field of view (FOV)

| Eyewear | Reference | Area | |
|---|---|---|---|
| Legacy 1 | 30° | 219 | cm² |
| Legacy 1 | 60° | 131 | cm² |
| Legacy 1 | 95° | 1 | cm² |
| Legacy 2 | 30° | 232 | cm² |
| Legacy 2 | 60° | 127 | cm² |
| Legacy 2 | 95° | 0 | cm² |
| Legacy 3 | 30° | 211 | cm² |
| Legacy 3 | 60° | 131 | cm² |
| Legacy 3 | 95° | 20 | cm² |
| Eyewear 126 | 30° | 331 | cm² |
| Eyewear 126 | 60° | 228 | cm² |
| Eyewear 126 | 95° | 29 | cm² |

In some aspects, a rate of change of a lens normal (wrap) of lens 100 is constant. In some aspects, the rate of change of the lens normal is a non-zero rate of change. In some aspects, the rate of change of the lens normal may be constant along at least a portion of lens body 110 along a vertical direction. The rate of change may be selected such that lens body 110 (e.g., the posterior surface of lens body 110) is near to a face of the wearer. In some aspects, the rate of change is greater than zero.

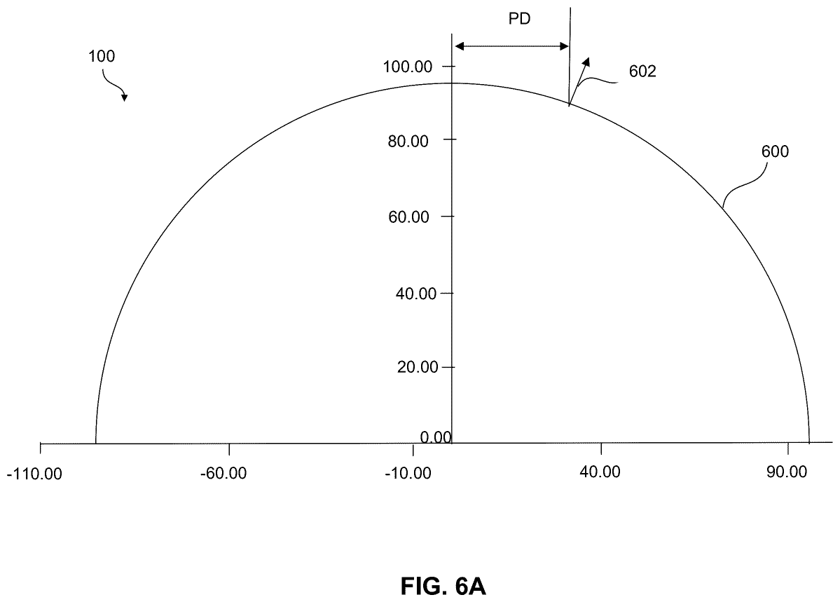

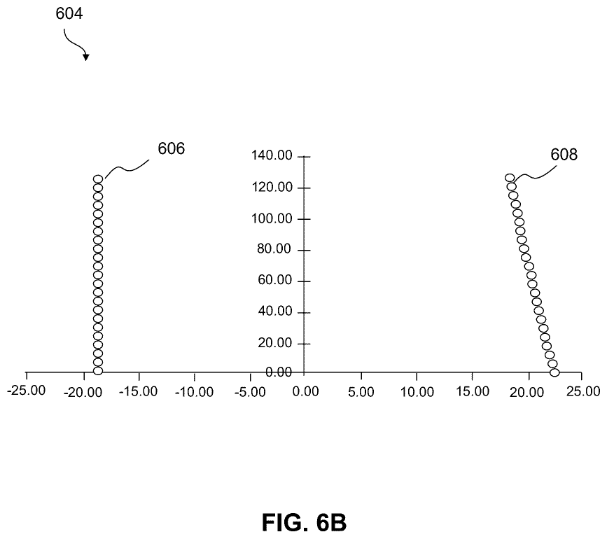

FIG. 6A is a schematic that illustrates a horizontal slice 600 of lens 100, according to some embodiments. Horizontal slice 600 may be obtained by taking a horizontal slice along a vertical direction. Different horizontal slices may be obtained and the normal at pupillary distance (PD) may be measured. The pupillary distance (PD) as used herein refers to the distance from the pupil of one eye to the mid-point between the eyes. In some aspects, the PD is 32 mm between the center of one eye and the center line (as shown in FIG. 6A). For each horizontal slice, a normal vector 602 may be determined. Each horizontal slice may have a different radius because lens 100 has a conical surface. The radius of each horizontal slice may decrease when obtaining the horizontal slices from the upper edge towards the lower edge of lens 100. In other embodiments, the radius may increase when obtaining slices from the upper edge to the lower edge (e.g., horizontal slice of shield 200 shown in FIG. 2B).

FIG. 6B is a graph 604 that illustrates wrap angles obtained from horizontal slices of a lens, according to some embodiments. Trace 606 shows the wrap angles of a cylindrical lens (i.e., a lens that has a surface that conforms to a surface of a cylinder). As shown from trace 606, the rate of change of the lens normal is zero. Trace 608 shows the wrap angles of a conical lens (e.g., lens 100). In graph 604, the y-axis represents the vertical distance along lens body 110 where y=0 may correspond to the upper or lower end of the lens (height). A rate of change in the lens normal or wrap angle is constant for the horizontal slices when moving from the top to the bottom of lens (or from the bottom to the top of the lens) (e.g., from upper edge 102 to lower edge 104 of lens 100).

In some aspects, an absolute value of the rate of change is greater than zero. The rate of change may be positive or negative. In some aspects, the rate of change may greater than about 0.01 degrees/mm, greater than about 0.02 degrees/mm, greater than about 0.03 degrees/mm, greater than about 0.04 degrees/mm, greater than about 0.05 degrees/mm. In some aspects, the rate of change may be greater than 0 degrees/mm and less than about 0.04 degrees/mm. In some aspects, the rate of change may be greater than 0 degrees/mm and less than about 0.035 degrees/mm. In some aspects, the rate of change may be greater than 0 degrees/mm and less than about 0.03 degrees/mm. In some aspects, the rate of change may be greater than 0 degrees/mm and less than about 0.025 degrees/mm. In some aspects, the rate of change may be greater than 0 degrees/mm and less than about 0.02 degrees/mm. In some aspects, the rate of change may be from about 0.01 degrees/mm to about 0.05 degrees/mm. In some aspects, the rate of change may be from about 0.01 degrees/mm to about 0.04 degrees/mm. In some aspects, the rate of change may be from about 0.01 degrees/mm to about 0.03 degrees/mm. In some aspects, the rate of change may be from about 0.01 degrees/mm to about 0.02 degrees/mm. In some aspects, the rate of change may be about 0.01 degrees/mm, about 0.02 degrees/mm, about 0.03 degrees/mm, about 0.04 degrees/mm, or about 0.05 degrees/mm.

### Differential Offset Measurements

In some aspects, a differential offset of lens 100 is greater than 6 mm. A differential offset may correspond to a maximum difference between any two horizontal distances between a surface of lens 100 and a vertical plane perpendicular to a pupillary axis of an EN headform.

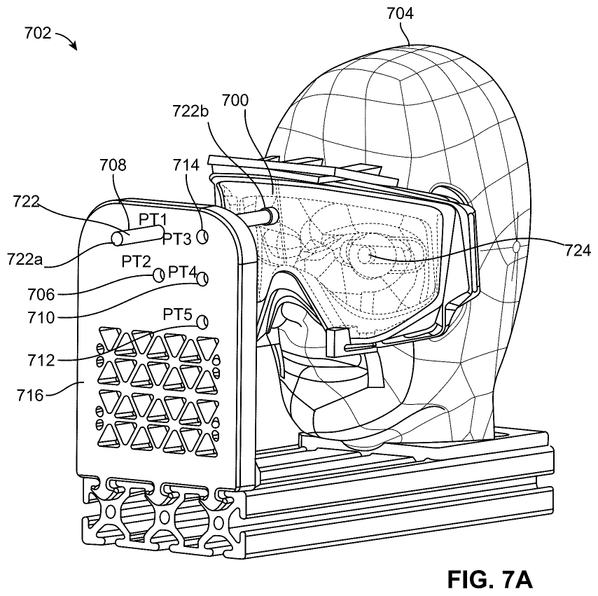

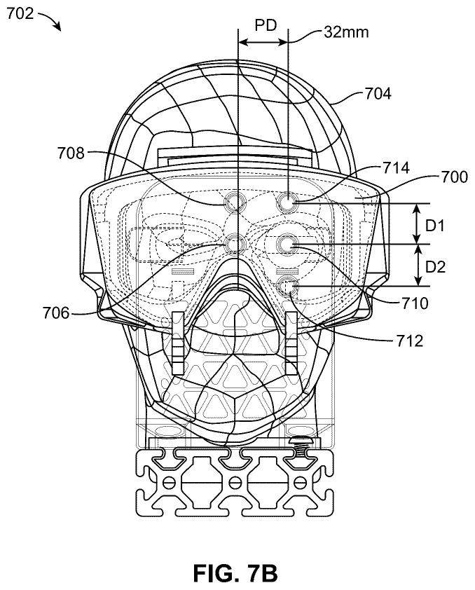

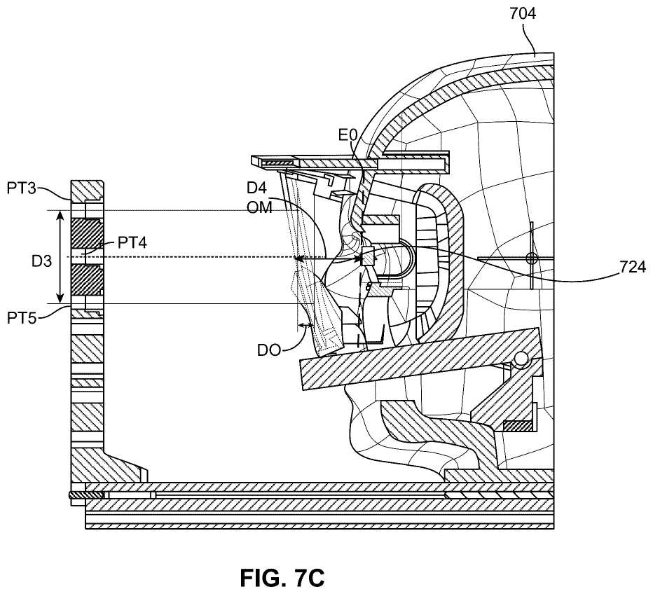

FIG. 7A is a schematic illustration of a perspective view of a differential offset measurement apparatus 702, according to an example embodiment. FIG. 7B shows a front view of differential offset measurement apparatus 702. A lens 700 is shown mounted on a EN headform 704. In some aspects, lens 100 may be positioned on EN headform 704 as described with respect to FIG. 3G (e.g., contact material is not compressed).

In some aspects, distances between an outer surface 700 of lens 100 to a vertical plane 716 may be determined. In some aspects, the distances may be determined relative to a predetermined distance. For example, vertical plane 716 may be fixed at the predetermined reference distance from the EN headform 704 such that a pin 722 corresponding to axis 710 (an axis that intersects the lens surface at PD) has its distal end 722a flush with vertical plane 716 and its proximal end 722b touches the EN headform's eyeball surface 724 on imaginary pupil reference vertical plane, E0 (illustrated in FIG. 7C). The distances from the outer surface 700 of lens 100 may be measured with respect to the pupil reference vertical plane E0. In some aspects, an “offset measurement”, OM, may refer to a distance between an outer surface 700 of lens 100 to pupil reference vertical plane E0. Such OM distance corresponds to the length of pin distal end 722a projecting from vertical plane 716. As such, when distal end 722a is flush with vertical plane 716, OM is 0, and when distal end 722a projects from vertical plane 716, OM is greater than 0 (the projecting amount representing the distance of outer surface 700 of lens 100 to imaginary pupil reference vertical plane, E0). As shown in FIG. 7C, for example, in some aspects, a distance D4 representing the OM from outer surface 700 of lens 100 to left eye surface 724 of EN headform 704 at axis 710 that intersects the lens surface at PD may be from about 30 mm to about 40 mm, from about 32 mm to about 38 mm, or from about 34 mm to about 36 mm.

In some aspects, distances may be determined along multiple axes. In some aspects, distances from outer surface of lens 700 may be measured along axis 708, axis 706, axis 710, axis 714, and axis 712. In some aspects, axis 710 may represent an axis that intersects the lens surface at PD. In the table below, PT1 represents the intersection of axis 708 with vertical plane 716, PT2 represents the intersection of axis 706 with vertical plane 716, PT3 represents the intersection of axis 714 with vertical plane 716, PT4 represents the intersection of axis 710 with vertical plane 716, and PT5 represents the intersection of axis 712 and vertical plane 716. PT3, PT4, and PT5 represent measurement at a PD distance (axis 714, axis 710, and axis 712 as shown in FIG. 7B). PT3 may be at a distance D1 from PT4. PT5 may be at a distance D2 from PT4. In some aspects, D1 may be equal to about 25 mm. D2 may be equal to about 25 mm. Table 2 shows offset measurements for a plurality of lenses at PT1, PT2, PT3, PT4, and PT5.

TABLE 2
Offset Measurements OM based on left
eyeball surface at E0 (in mm)

| | | | | | | Differential offset: |
|---|---|---|---|---|---|---|
| Lens | PT. 1 | PT. 2 | PT. 3 | PT. 4 | PT. 5 | PT. 3 − PT. 5 |
| Lens 100 | 46.91 | 41.17 | 40.54 | 34.7 | 29.11 | 11.43 |
| Legacy 1 | 54.73 | 53.21 | 47.86 | 45.88 | 42.07 | 5.79 |
| Legacy 2 | 50.38 | 48.11 | 43.95 | 41.91 | 40.14 | 3.8 |
| Legacy 3 | 51.18 | 51.09 | 44.59 | 44.54 | 42.86 | 1.73 |
| Legacy 4 | 55.96 | 55.99 | 49.68 | 49.25 | 48.31 | 1.37 |
| Legacy 5 | 53.08 | 55.21 | 46.46 | 48.77 | 44.66 | 1.8 |
| Legacy 6 | 56.68 | 54.97 | 50.08 | 48.59 | 47.44 | 2.64 |
| Legacy 7 | 58.71 | 59.96 | 51.54 | 53.46 | 50.47 | 1.07 |
| Legacy 8 | 49.76 | 48.58 | 44.08 | 42.72 | 41.77 | 2.31 |

FIG. 7C is a schematic illustration of a side view of differential offset measurement apparatus 702, according to some embodiments. In some aspects, a differential offset (DO shown in FIG. 7C) may be defined as the maximum between two offset measurements within a vertical range D3. In some aspects, D3 may be between about 40 mm to about 60 mm or between about 45 mm to about 55 mm. In some aspects, D3 is equal to about 50 mm.

In some aspects, a differential offset of lens 100 may be greater than about 6 mm. In some aspects, the differential offset may be greater than about 7 mm, greater than about 8 mm, greater than about 9 mm, greater than about 10 mm, greater than about 12 mm, or greater than about 13 mm. In some aspects, the differential offset may be from about 6 mm to about 15 mm, from about 7 mm to about 14 mm, or from about 8 mm to about 13 mm. In some aspects, the differential offset is equal to about 7 mm, to about 8 mm, to about 9 mm, to about 10 mm, to about 11 mm, or to about 12 mm. Table 2 shows differential offset measurements between PT3 and PT5 for a plurality of lenses.

In some aspects, a differential offset of lens 100 between PT3 and PT5 may be greater than about 6 mm. In some aspects, the differential offset may be greater than about 7 mm, greater than about 8 mm, greater than about 9 mm, greater than about 10 mm, greater than about 12 mm, or greater than about 13 mm. In some aspects, the differential offset may be from about 6 mm to about 15 mm, from about 7 mm to about 14 mm, or from about 8 mm to about 13 mm. In some aspects, the differential offset is equal to about 7 mm, to about 8 mm, to about 9 mm, to about 10 mm, to about 11 mm, or to about 12 mm.

The foregoing disclosure outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A lens for an eyewear, comprising:
a portion of a conical surface configured to be located in the path of a wearer's field of vision, wherein:
the portion of the conical surface has a first side edge and a second side edge;
the portion of the conical surface extends arcuately from the first side edge to the second side edge;
the portion of the conical surface has an upper edge and a lower edge;
the portion of the conical surface extends from the upper edge to the lower edge;
a non-zero rate of change of a lens normal from the upper edge to the lower edge is constant; and
the lens normal is measured along an axis that intersects the conical surface at a pupillary distance.

2. The lens of claim 1, wherein an absolute value of the non-zero rate of change of the lens normal is between about 0.01 to about 0.13 degrees/mm.

3. The lens of claim 2, wherein the non-zero rate of change of the lens normal is between about 0.01 to about 0.13 degrees/mm.

4. The lens of claim 1, wherein an absolute value of the non-zero rate of change of the lens normal is greater than about 0.03 degrees/mm.

5. The lens of claim 4, wherein the non-zero rate of change of the lens normal is greater than about 0.03 degrees/mm.

6. The lens of claim 1, wherein a perpendicular distance between the upper edge to the lower edge is from about 100 mm to about 130 mm.

7. The lens of claim 1, wherein:
a differential offset of the lens is greater than 6 mm;
the differential offset is measured using an EN headform;
the differential offset corresponds to a maximum difference between any two horizontal distances from the portion of the conical surface to a vertical plane;
the vertical plane is perpendicular to a pupillary axis from the EN headform; and
the vertical plane is positioned in front of the EN headform.

8. The lens of claim 7, wherein the vertical plane in an imaginary pupil reference vertical plane positioned at an eyeball surface of the EN headform.

9. The lens of claim 7, wherein the differential offset is between about 8 mm and about 12 mm.

10. The lens of claim 9, wherein the horizontal distances are measured within a vertical range of about −25 mm to about +25 mm from an intersection of the pupillary axis with the vertical plane.

11. The lens of claim 1, wherein:
an area of a shadow of the lens that is outside of a first field of view and to the left of an eye center axis of a left eye of an EN headform greater than 300 cm²;
the first field of view is the intersection of a 30° light cone emanating from an eye center of the EN headform with a cylindrical target;
the shadow identifies the extent of the lens as cast on the cylindrical target; and
a radius of a cylinder defining a surface of the cylindrical target is 243 mm and a distance between the eye center of the EN headform and the cylindrical target is 73 mm.

12. An eyewear comprising the lens of claim 1.

13. The eyewear of claim 12, wherein the eyewear is a goggle.

14. A shield comprising the lens of claim 1, wherein the non-zero rate of change of the lens normal is negative.

15. A lens, comprising:
a portion of a conical surface configured to be located in a path of a wearer's field of vision, wherein:
the portion of the conical surface extends arcuately from a first side edge to a second side edge;
the first side edge and the second side edge have a substantially vertical direction when the conical surface is located in the path of the wearer's field of vision;
the portion of the conical surface extends between an upper edge and a lower edge;
a differential offset of the portion of the conical surface is greater than about 6 mm;
the differential offset is measured using an EN headform;
the differential offset corresponds to a maximum difference between any two horizontal distances between the portion of the conical surface and a vertical plane;
the vertical plane is perpendicular to a pupillary axis from the EN headform; and
the vertical plane is positioned in front of the EN headform.

16. The lens of claim 15, wherein the vertical plane in an imaginary pupil reference vertical plane positioned at an eyeball surface of the EN headform.

17. The lens of claim 15, wherein the differential offset is between about 8 mm and about 12 mm.

18. The lens of claim 15, wherein the horizontal distances are measured within a vertical range of about −25 mm to about +25 mm from an intersection of the pupillary axis with the vertical plane.

19. The lens of claim 15, wherein:
a non-zero rate of change of a lens normal from the upper edge to the lower edge is constant; and
the lens normal is measured along an axis that intersects the conical surface at a pupillary distance.

20. The lens of claim 15, wherein:
an area of a shadow of the lens that is outside of a first field of view and to the left of an eye center axis of a left eye of the EN headform is greater than 300 cm²;
the first field of view is the intersection of a 30° light cone emanating from an eye center of an EN headform with a cylindrical target;
the shadow identifies the extent of the lens as cast on the cylindrical target; and
a radius of a cylinder defining a surface of the cylindrical target is 243 mm and a distance between the eye center of the EN headform and the cylindrical target is 73 mm.

21. An eyewear comprising the lens of claim 15.

22. The eyewear of claim 21, wherein the eyewear is a goggle.

23. An eyewear comprising:
a frame configured to be worn conformally on a user's face; and
a lens coupled to the frame, wherein:
a surface of the lens substantially conforms to a portion of a surface of a cone;
the lens extends arcuately from a first linear side edge to a second linear side edge;
the lens extends from an upper edge to a lower edge;
the upper edge extends arcuately from a first end of the first linear side edge to a first end of the second linear side edge;
the upper edge includes an upper arc segment, the upper arc segment being part of a first ellipse;
the lower edge include a lower arc segment, the lower arc segment being part of a second ellipse; and
a semi-minor axis of the first ellipse is greater than a semi-minor axis of the second ellipse.

24. The eyewear of claim 23, wherein the cone has a circular base.

25. The eyewear of claim 23, wherein the radius of the upper arc segment is between about 80 mm to 120 mm.

26. The eyewear of claim 23, wherein the radius of the lower arc segment is between about 60 mm to about 100 mm.

27. The eyewear of claim 23, wherein the lower edge extends arcuately from a second end of the first linear side edge to a second end of the second linear side edge.

28. The eyewear of claim 23, wherein a non-zero rate of change of a lens normal from the upper edge to the lower edge is constant; and
wherein the lens normal is measured along an axis that intersects the conical surface at a pupillary distance.

29. The eyewear of claim 28, wherein a perpendicular distance between the upper edge to the lower edge is from about 100 mm to about 130 mm.

30. The eyewear of claim 23, wherein the eyewear is a goggle.
